(19) United States
(12) Patent Application Publication
(10) Pub. No.: US 2021/0313027 A1
(43) Pub. Date: Oct. 7, 2021

(54) SYSTEM AND METHOD FOR VISUALIZING THE ELECTRONIC HEALTH RECORD USING A PATTERNED TIMELINE

(71) Applicant: David Clarkson, Fairhope, AL (US)

(72) Inventor: David Clarkson, Fairhope, AL (US)

(21) Appl. No.: 16/838,601

(22) Filed: Apr. 2, 2020

(51) Int. Cl. G16H 15/00; G16H 10/60; G06F 16/25; G06F 16/248

(52) U.S. Cl. CPC G16H 15/00; G06F 16/248; G06F 16/258; G16H 10/60

(57) ABSTRACT

A system and method for creating a patterned timeline that is representative of a patient's medical history is provided. The system generally comprises a processor, a power supply, a display operably connected to the processor, a non-transitory computer-readable medium coupled to the processor and having instructions stored thereon, and a database operably connected to the processor. The processor may query the non-transitory computer-readable medium and/or database for patient health records and parse the patient health records for event data, which may be used to create medical events. The processor may then may then create patterned timelines within the user interface using the medical events so that a healthcare professional may visually navigate a patient's medical history.

### FIELD OF THE DISCLOSURE

The subject matter of the present disclosure refers generally to a system and method for deconstructing and visualizing the electronic health record.

### BACKGROUND

Prior to seeing a patient, healthcare professionals must review a patient's health records within the electronic health record (EHR) in order to acquire an understanding of that patient's medical history. Without this knowledge, a healthcare professional might make a poorly formed diagnosis made using only part of the knowledge available. Unfortunately, the EHR can be very slow to navigate, and depending on the size of the patient's medical history, it can take some time to review all of the information within relevant to the patient. This is partly due to the fact that as much as 75% of notes in any given patient medical record are copy and pasted from a previous documented medical event. For patients having extensive medical histories, this may result in a single medical record having multiple screens worth of notes that are not necessarily relevant to the patient's most recent visit, which greatly decreases the efficiency in which a healthcare professional can address the patient's needs. Additionally, the sorting and filing system used for written History and Physicals (H&P), which comprises a series of cabinets with drawers and sub-files, is more or less a more searchable digitized version of the Dewey decimal system used in libraries more than seventy-five years ago. This can make it particularly difficult for healthcare professionals to search a patient's health record for a particular piece of information, which can greatly slow down the efficiency in which a healthcare professional can work.

Not only is the slow evolution of the EHR potentially hazardous to patient health due to increased potential of missed information, but it also increases costs by decreasing efficiency. Furthermore, the current EHR overloads healthcare professionals with information, which can obscure what underlying problem a patient may be dealing with. Information overload can also cause burnout due to the difficulty of using EHRs and reviewing the unnecessarily large amount of data often provided to healthcare professionals when reviewing a patient's medical history. Burnout may also cause healthcare professionals to become careless, which is detrimental to patient safety.

Therefore, there is a need in the art for a system and method that transforms patient medical records of the EHR into a patterned timeline of a patient's medical history in order to maximize technological innovation, and therefore, increase efficiency of healthcare professionals.

### SUMMARY

A system and method for transforming the EHR into a patterned timeline is disclosed herein. The method generally comprises visualizing a patient's EHR by extracting the most important data and spatially ordering this data about a timeline that proceeds vertically, horizontally, or diagonally, depending on the time between medical events. The pattern created by the timeline may indicate more to a healthcare professional in a moment than several minutes of review of the EHR may otherwise reveal. The system generally comprises a processor, a power supply, a display operably connected to the processor, and a non-transitory computer-readable medium coupled to the processor and having instructions stored thereon. In one embodiment, a computing device having a user interface may be operably connected to the processor. In another embodiment, a database operably connected to the processor may be used to store medical events in patient profiles, wherein said medical events are obtained by parsing patient health records. In yet another embodiment, a server may be operably connected to the database and processor, facilitating the transfer of information between the processor and database. It is understood that the various method steps associated with the methods of the present disclosure may be carried out as operations by the system. Various permission levels may be utilized by the system for controlling access to the data contained within.

The system may be used to carry out the methods described herein by parsing the EHR into event data, reconstructing event data into medical events, and creating a patterned timeline of the medical events within the user interface, which may then be presented via a display to a healthcare professional. The processor may begin by querying the non-transitory computer-readable medium and/or database for patient profiles having event data and/or medical events. If no patient profiles having event data and/or medical events are found, the processor may search the EHR for a patient's health records and parse said health records for event data, which the system may then use to create medical events. In one preferred embodiment, the processor may perform a check to determine if at least one data filter has been selected within the user interface. If the processor determines that at least one data filter has been selected within the user interface, the processor may filter medical events of the system based on the chosen search parameters. After filtering the medical events, the processor may plot the medical events about a patterned timeline within the user interface. In one preferred embodiment, the user may alter the way in which the medical events are ordered about the patterned timeline by changing pattern parameters within the user interface.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTON OF THE DRAWINGS

These and other features, aspects, and advantages of the present disclosure will become better understood with regard to the following description, appended claims, and accompanying drawings where:

FIG. 1 is a diagram of an example environment in which techniques described herein may be implemented.

FIG. 2 is a diagram of an example environment in which techniques described herein may be implemented.

FIG. 3 is a diagram of an example environment in which techniques described herein may be implemented.

FIG. 4 is a diagram illustrating a system embodying features consistent with the principles of the present disclosure.

FIG. 5 is an illustration of a preferred embodiment of the user interface.

FIG. 6 is a diagram illustrating the manner in which individual access to data may be granted or limited based on user roles or administrator roles.

FIG. 7 is a flow chart illustrating certain method steps of a method embodying features consistent with the principles of the present disclosure.

FIG. 8 is a flow chart illustrating certain method steps of a method embodying features consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, steps, etc. are optionally present. For example, a system “comprising” components A, B, and C can contain only components A, B, and C, or can contain not only components A, B, and C, but also one or more other components. The term “parse” and grammatical equivalents thereof are used herein to mean to parse and/or to perform a lexical analysis. The term “deconstructs” and grammatical equivalents thereof are used herein to mean the act of breaking something down into its separate parts in order to understand its meaning. In regards to patient data, deconstruction is meant to indicate the process whereby the data of the electronic health record (EHR) is broken down and re-combined to create medical events. This would facilitate the review of the patient data in isolation from the other disparate portions. As used herein, the term “database” refers to a set of related data and the way it is organized. Access to this data is usually provided by a database management system (DBMS) consisting of an integrated set of computer software that allows healthcare professionals to interact with one or more databases and provides access to all of the data contained in the database. The DBMS provides various functions that allow entry, storage and retrieval of large quantities of information and provides ways to manage how that information is organized. Because of the close relationship between the database and the DBMS, as used herein, the term “database” refers to both a database and DBMS.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility). As will be evident from the disclosure provided below, the present invention satisfies the need for a system and method capable of managing and transforming data related to the electronic health record (EHR), and thereby improving upon known systems currently employed within the art.

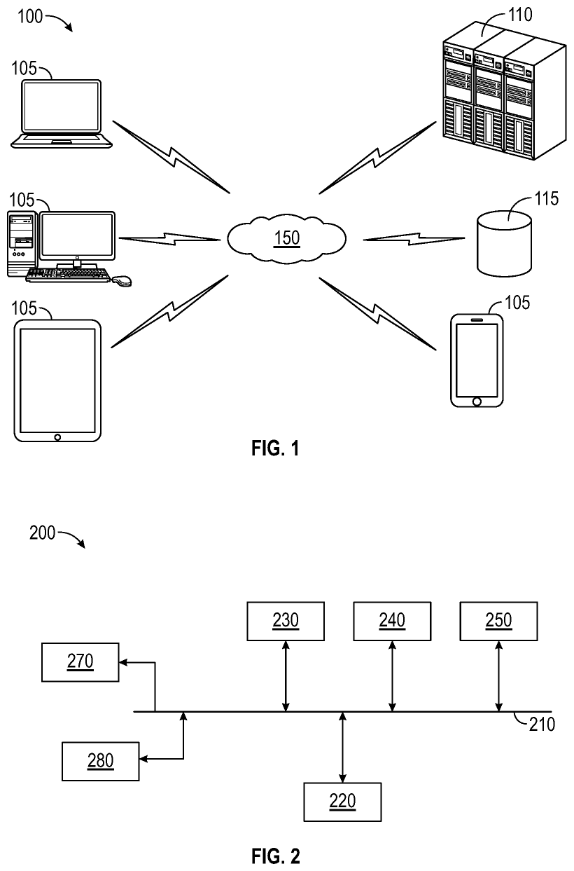

FIG. 1 depicts an exemplary environment 100 of the system 400 consisting of clients 105 connected to a server 110 and/or database 115 via a network 150. Clients 105 are devices of users 405 that may be used to access servers 110 and/or databases 115 through a network 150. A network 150 may comprise of one or more networks of any kind, including, but not limited to, a local area network (LAN), a wide area network (WAN), metropolitan area networks (MAN), a telephone network, such as the Public Switched Telephone Network (PSTN), an intranet, the Internet, a memory device, another type of network, or a combination of networks. In a preferred embodiment, computing entities 200 may act as clients 105 for a user 405. For instance, a client 105 may include a personal computer, a wireless telephone, a personal digital assistant (PDA), a laptop, a smart phone, a tablet computer, or another type of computation or communication device. Servers 110 may include devices that access, fetch, aggregate, process, search, provide, and/or maintain documents. Although FIG. 1 depicts a preferred embodiment of an environment 100 for the system 400, in other implementations, the environment 100 may contain fewer components, different components, differently arranged components, and/or additional components than those depicted in FIG. 1. Alternatively, or additionally, one or more components of the environment 100 may perform one or more other tasks described as being performed by one or more other components of the environment 100.

As depicted in FIG. 1, one embodiment of the system 400 may comprise a server 110. Although shown as a single server 110 in FIG. 1, a server 110 may, in some implementations, be implemented as multiple devices interlinked together via the network 150, wherein the devices may be distributed over a large geographic area and performing different functions or similar functions. For instance, two or more servers 110 may be implemented to work as a single server 110 performing the same tasks. Alternatively, one server 110 may perform the functions of multiple servers 110. For instance, a single server 110 may perform the tasks of a web server and an indexing server. Additionally, it is understood that multiple servers 110 may be used to operably connect the processor 220 to the database 115 and/or other content repositories. The processor 220 may be operably connected to the server 110 via wired or wireless connection. Types of servers 110 that may be used by the system 400 include, but are not limited to, search servers, document indexing servers, and web servers, or any combination thereof.

Search servers may include one or more computing entities 200 designed to implement a search engine, such as a documents/records search engine, general webpage search engine, etc. Search servers may, for example, include one or more web servers designed to receive search queries and/or inputs from users 405, search one or more databases 115 in response to the search queries and/or inputs, and provide documents or information, relevant to the search queries and/or inputs, to users 405. In some implementations, search servers may include a web search server that may provide webpages to users 405, wherein a provided webpage may include a reference to a web server at which the desired information and/or links are located. The references to the web server at which the desired information is located may be included in a frame and/or text box, or as a link to the desired information/document. Document indexing servers may include one or more devices designed to index documents available through networks 150.

Document indexing servers may access other servers 110, such as web servers that host content, to index the content. In some implementations, document indexing servers may index documents/records stored by other servers 110 connected to the network 150. Document indexing servers may, for example, store and index content, information, and documents relating to user accounts and user-generated content. Web servers may include servers 110 that provide webpages to clients 105. For instance, the webpages may be HTML-based webpages. A web server may host one or more websites. As used herein, a website may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

FIG. 2 is an exemplary diagram of a client 105, server 110, computing device, and/or or database 115 (hereinafter collectively referred to as “computing entity 200”), which may correspond to one or more of the clients 105, servers 110, and databases 115 according to an implementation consistent with the principles of the invention as described herein. The computing entity 200 may comprise a bus 210, a processor 220, memory 304, a storage device 250, a peripheral device 270, and a communication interface 280. The bus 210 may be defined as one or more conductors that permit communication among the components of the computing entity 200. The processor 220 may be defined as a logic circuitry that responds to and processes the basic instructions that drive the computing entity 200. Memory 304 may be defined as the integrated circuitry that stores information for immediate use in a computing entity 200. A peripheral device 270 may be defined as any hardware used by a user 405 and/or the computing entity 200 to facilitate communicate between the two. A storage device 250 may be defined as a device used to provide mass storage to a computing entity 200. A communication interface 280 may be defined as any transceiver-like device that enables the computing entity 200 to communicate with other devices and/or computing entities 200.

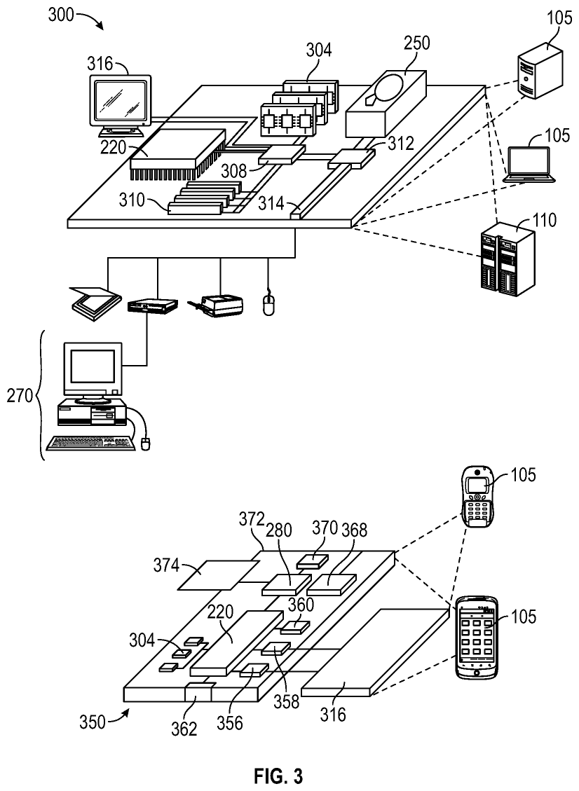

The bus 210 may comprise a high-speed interface 308 and/or a low-speed interface 312 that connects the various components together in a way such they may communicate with one another, as illustrated in the computing device of FIG. 3. A high-speed interface 308 manages bandwidth-intensive operations for computing device, while a low-speed interface 312 manages lower bandwidth-intensive operations. In some preferred embodiments, the high-speed interface 308 of a bus 210 may be coupled to the memory 304, display 316, and to high-speed expansion ports 310, which may accept various expansion cards such as a graphics processing unit (GPU). In other preferred embodiments, the low-speed interface 312 of a bus 210 may be coupled to a storage device 250 and low-speed expansion ports 314. The low-speed expansion ports 314 may include various communication ports, such as USB, Bluetooth, Ethernet, wireless Ethernet, etc. Additionally, the low-speed expansion ports 314 may be coupled to one or more peripheral devices 270, such as a keyboard, pointing device, scanner, and/or a networking device, wherein the low-speed expansion ports 314 facilitate the transfer of input data from the peripheral devices 270 to the processor 220 via the low-speed interface 312.

The processor 220 may comprise any type of conventional processor or microprocessor that interprets and executes computer readable instructions. The processor 220 is configured to perform the operations disclosed herein based on instructions stored within the system 400. The processor 220 may process instructions for execution within the computing entity 200, including instructions stored in memory 304 or on a storage device 250, to display graphical information for a graphical user interface (GUI) on an external peripheral device 270, such as a display 316. The processor 220 may provide for coordination of the other components of a computing entity 200, such as control of user interfaces 411, applications run by a computing entity 200, and wireless communication by a communication device of the computing entity 200. The processor 220 may be any processor or microprocessor suitable for executing instructions. In some embodiments, the processor 220 may have a memory device therein or coupled thereto suitable for storing the data, content, or other information or material disclosed herein. In some instances, the processor 220 may be a component of a larger computing entity 200. A computing entity 200 that may house the processor 220 therein may include, but are not limited to, laptops, desktops, workstations, personal digital assistants, servers 110, mainframes, cellular telephones, tablet computers, or any other similar device. Accordingly, the inventive subject matter disclosed herein, in full or in part, may be implemented or utilized in devices including, but are not limited to, laptops, desktops, workstations, personal digital assistants, servers 110, mainframes, cellular telephones, tablet computers, or any other similar device.

Memory 304 stores information within a computing entity 200, as illustrated in the computing device 300 of FIG. 3. In some preferred embodiments, memory 304 may include one or more volatile memory units. In another preferred embodiment, memory 304 may include one or more non-volatile memory units. Memory 304 may also include another form of computer-readable medium, such as a magnetic or optical disk. For instance, a portion of a magnetic hard drive may be partitioned as a dynamic scratch space to allow for temporary storage of information that may be used by the processor 220 when faster types of memory, such as random-access memory (RAM), are in high demand. A computer-readable medium may refer to a non-transitory computer-readable memory device. A memory device may refer to storage space within a single storage device 250 or spread across multiple storage devices 250. The memory 304 may comprise main memory 230 and/or read only memory (ROM) 240. In a preferred embodiment, the main memory 230 may comprise RAM or another type of dynamic storage device 250 that stores information and instructions for execution by the processor 220. ROM 240 may comprise a conventional ROM device or another type of static storage device 250 that stores static information and instructions for use by processor 220. The storage device 250 may comprise a magnetic and/or optical recording medium and its corresponding drive.

As mentioned earlier, a peripheral device 270 is a device that facilitates communication between a user 405 and the processor 220. The peripheral device 270 may include, but is not limited to, an input device and/or an output device. As used herein, an input device may be defined as a device that allows a user 405 to input data and instructions that is then converted into a pattern of electrical signals in binary code that are comprehensible to a computing entity 200. An input device of the peripheral device 270 may include one or more conventional devices that permit a user 405 to input information into the computing entity 200, such as a scanner, phone, camera, scanning device, keyboard, a mouse, a pen, voice recognition and/or biometric mechanisms, etc. As used herein, an output device may be defined as a device that translates the electronic signals received from a computing entity 200 into a form intelligible to the user 405. An output device of the peripheral device 270 may include one or more conventional devices that output information to a user 405, including a display 316, a printer, a speaker, an alarm, a projector, etc. Additionally, storage devices 250, such as CD-ROM drives, and other computing entities 200 may act as a peripheral device 270 that may act independently from the operably connected computing entity 200. For instance, a fitness tracker may transfer data to a smartphone, wherein the smartphone may use that data in a manner separate from the fitness tracker.

The storage device 250 is capable of providing the computing entity 200 mass storage. In some embodiments, the storage device 250 may comprise a computer-readable medium such as the memory 304, storage device 250, or memory 304 on the processor 220. A computer-readable medium may be defined as one or more physical or logical memory devices and/or carrier waves. Devices that may act as a computer readable medium include, but are not limited to, a hard disk device, optical disk device, tape device, flash memory or other similar solid-state memory device, or an array of devices, including devices in a storage area network or other configurations. Examples of computer-readable mediums include, but are not limited to, magnetic media such as hard disks, floppy disks, and magnetic tape; optical media such as CD ROM discs and DVDs; magneto-optical media such as optical discs; and hardware devices that are specially configured to store and perform programming instructions, such as ROM 240, RAM, flash memory, and the like.

In an embodiment, a computer program may be tangibly embodied in the storage device 250. The computer program may contain instructions that, when executed by the processor 220, performs one or more steps that comprise a method, such as those methods described herein. The instructions within a computer program may be carried to the processor 220 via the bus 210. Alternatively, the computer program may be carried to a computer-readable medium, wherein the information may then be accessed from the computer-readable medium by the processor 220 via the bus 210 as needed. In a preferred embodiment, the software instructions may be read into memory 304 from another computer-readable medium, such as data storage device 250, or from another device via the communication interface 280. Alternatively, hardwired circuitry may be used in place of or in combination with software instructions to implement processes consistent with the principles as described herein. Thus, implementations consistent with the invention as described herein are not limited to any specific combination of hardware circuitry and software.

FIG. 3 depicts exemplary computing entities 200 in the form of a computing device 300 and mobile computing device 350, which may be used to carry out the various embodiments of the invention as described herein. A computing device 300 is intended to represent various forms of digital computers, such as laptops, desktops, workstations, servers 110, databases, mainframes, and other appropriate computers. A mobile computing device 350 is intended to represent various forms of mobile devices, such as scanners, scanning devices, personal digital assistants, cellular telephones, smart phones, tablet computers, and other similar devices. The various components depicted in FIG. 3, as well as their connections, relationships, and functions are meant to be examples only, and are not meant to limit the implementations of the invention as described herein. The computing device 300 may be implemented in a number of different forms, as shown in FIGS. 1 and 3. For instance, a computing device 300 may be implemented as a server 110 or in a group of servers 110. Computing devices 300 may also be implemented as part of a rack server system. In addition, a computing device 300 may be implemented as a personal computer, such as a desktop computer or laptop computer. Alternatively, components from a computing device 300 may be combined with other components in a mobile device, thus creating a mobile computing device 350. Each mobile computing device 350 may contain one or more computing devices 300 and mobile devices, and an entire system may be made up of multiple computing devices 300 and mobile devices communicating with each other as depicted by the mobile computing device 350 in FIG. 3. The computing entities 200 consistent with the principles of the invention as disclosed herein may perform certain receiving, communicating, generating, output providing, correlating, and storing operations as needed to perform the various methods as described in greater detail below.

In the embodiment depicted in FIG. 3, a computing device 300 may include a processor 220, memory 304 a storage device 250, high-speed expansion ports 310, low-speed expansion ports 314, and bus 210 operably connecting the processor 220, memory 304, storage device 250, high-speed expansion ports 310, and low-speed expansion ports 314. In one preferred embodiment, the bus 210 may comprise a high-speed interface 308 connecting the processor 220 to the memory 304 and high-speed expansion ports 310 as well as a low-speed interface 312 connecting to the low-speed expansion ports 314 and the storage device 250. Because each of the components are interconnected using the bus 210, they may be mounted on a common motherboard as depicted in FIG. 3 or in other manners as appropriate. The processor 220 may process instructions for execution within the computing device 300, including instructions stored in memory 304 or on the storage device 250. Processing these instructions may cause the computing device 300 to display graphical information for a GUI on an output device, such as a display 316 coupled to the high-speed interface 308. In other implementations, multiple processors and/or multiple buses may be used, as appropriate, along with multiple memory units and/or multiple types of memory. Additionally, multiple computing devices 300 may be connected, wherein each device provides portions of the necessary operations.

A mobile computing device 350 may include a processor 220, memory 304 a peripheral device 270 (such as a display 316, a communication interface 280, and a transceiver 368, among other components). A mobile computing device 350 may also be provided with a storage device 250, such as a micro-drive or other previously mentioned storage device 250, to provide additional storage. Preferably, each of the components of the mobile computing device 350 are interconnected using a bus 210, which may allow several of the components of the mobile computing device 350 to be mounted on a common motherboard as depicted in FIG. 3 or in other manners as appropriate. In some implementations, a computer program may be tangibly embodied in an information carrier. The computer program may contain instructions that, when executed by the processor 220, perform one or more methods, such as those described herein. The information carrier is preferably a computer- readable medium, such as memory, expansion memory 374, or memory 304 on the processor 220 such as ROM 240, that may be received via the transceiver or external interface 362. The mobile computing device 350 may be implemented in a number of different forms, as shown in FIG. 3. For example, a mobile computing device 350 may be implemented as a cellular telephone, part of a smart phone, personal digital assistant, or other similar mobile device.

The processor 220 may execute instructions within the mobile computing device 350, including instructions stored in the memory 304 and/or storage device 250. The processor 220 may be implemented as a chipset of chips that may include separate and multiple analog and/or digital processors. The processor 220 may provide for coordination of the other components of the mobile computing device 350, such as control of the user interfaces 411, applications run by the mobile computing device 350, and wireless communication by the mobile computing device 350. The processor 220 of the mobile computing device 350 may communicate with a user 405 through the control interface 358 coupled to a peripheral device 270 and the display interface 356 coupled to a display 316. The display 316 of the mobile computing device 350 may include, but is not limited to, Liquid Crystal Display (LCD), Light Emitting Diode (LED) display, Organic Light Emitting Diode (OLED) display, and Plasma Display Panel (PDP), or any combination thereof. The display interface 356 may include appropriate circuitry for causing the display 316 to present graphical and other information to a user 405. The control interface 358 may receive commands from a user 405 via a peripheral device 270 and convert the commands into a computer readable signal for the processor 220. In addition, an external interface 362 may be provided in communication with processor 220, which may enable near area communication of the mobile computing device 350 with other devices. The external interface 362 may provide for wired communications in some implementations or wireless communication in other implementations. In a preferred embodiment, multiple interfaces may be used in a single mobile computing device 350 as is depicted in FIG. 3.

Memory 304 stores information within the mobile computing device 350. Devices that may act as memory 304 for the mobile computing device 350 include, but are not limited to computer-readable media, volatile memory, and non-volatile memory. Expansion memory 374 may also be provided and connected to the mobile computing device 350 through an expansion interface 372, which may include a Single In-Line Memory Module (SIM) card interface or micro secure digital (Micro-SD) card interface. Expansion memory 374 may include, but is not limited to, various types of flash memory and non-volatile random-access memory (NVRAM). Such expansion memory 374 may provide extra storage space for the mobile computing device 350. In addition, expansion memory 374 may store computer programs or other information that may be used by the mobile computing device 350. For instance, expansion memory 374 may have instructions stored thereon that, when carried out by the processor 220, cause the mobile computing device 350 perform the methods described herein. Further, expansion memory 374 may have secure information stored thereon; therefore, expansion memory 374 may be provided as a security module for a mobile computing device 350, wherein the security module may be programmed with instructions that permit secure use of a mobile computing device 350. In addition, expansion memory 374 having secure applications and secure information stored thereon may allow a user 405 to place identifying information on the expansion memory 374 via the mobile computing device 350 in a non-hackable manner.

A mobile computing device 350 may communicate wirelessly through the communication interface 280, which may include digital signal processing circuitry where necessary. The communication interface 280 may provide for communications under various modes or protocols, including, but not limited to, Global System Mobile Communication (GSM), Short Message Services (SMS), Enterprise Messaging System, Multimedia Messaging Service (MMS), Code Division Multiple Access (CDMA), Time Division Multiple Access (TDMA), Personal Digital Cellular (PDC), Wideband Code Division Multiple Access (WCDMA), IMT Multi-Carrier (CDMAX 0), and General Packet Radio Service (GPRS), or any combination thereof. Such communication may occur, for example, through a transceiver 368. Short-range communication may occur, such as using a Bluetooth, WIFI, or other such transceiver 368. In addition, a Global Positioning System (GPS) receiver module 370 may provide additional navigation-and location-related wireless data to the mobile computing device 350, which may be used as appropriate by applications running on the mobile computing device 350. Alternatively, the mobile computing device 350 may communicate audibly using an audio codec 360, which may receive spoken information from a user 405 and covert the received spoken information into a digital form that may be processed by the processor 220. The audio codec 360 may likewise generate audible sound for a user 405, such as through a speaker, e.g., in a handset of mobile computing device 350. Such sound may include sound from voice telephone calls, recorded sound such as voice messages, music files, etc. Sound may also include sound generated by applications operating on the mobile computing device 350.

The system 400 may also comprise a power supply. The power supply may be any source of power that provides the system 400 with power. In an embodiment, the power supply may be a stationary power outlet. The system 400 may comprise of multiple power supplies that may provide power to the system 400 in different circumstances. For instance, the system 400 may be directly plugged into a stationary power outlet, which may provide power to the system 400 so long as it remains in one place. However, the system 400 may also be connected to a backup battery so that the system 400 may receive power even when the it is not connected to a stationary power outlet or if the stationary power outlet ceases to provide power to the computing entity 200.

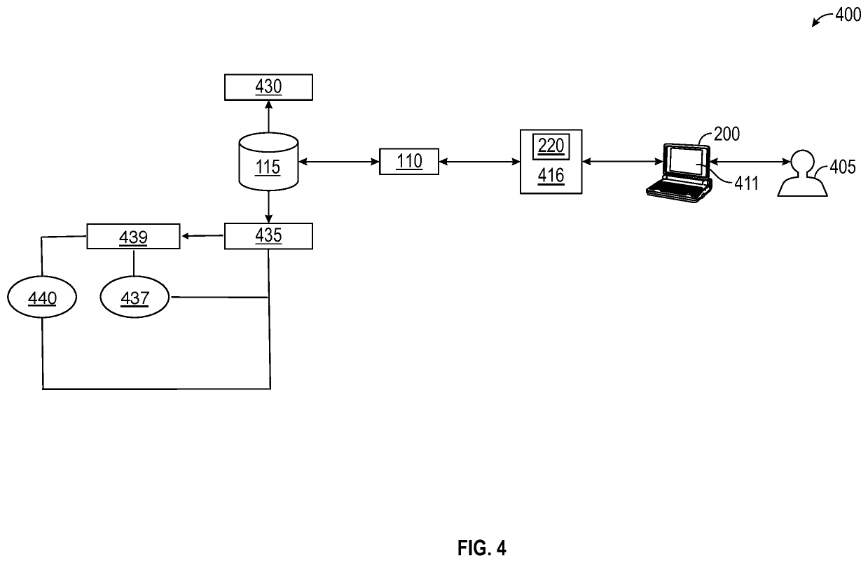

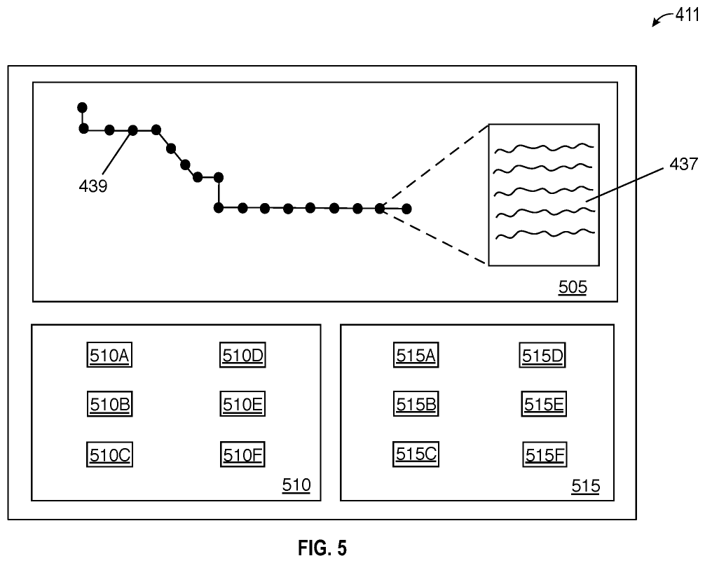

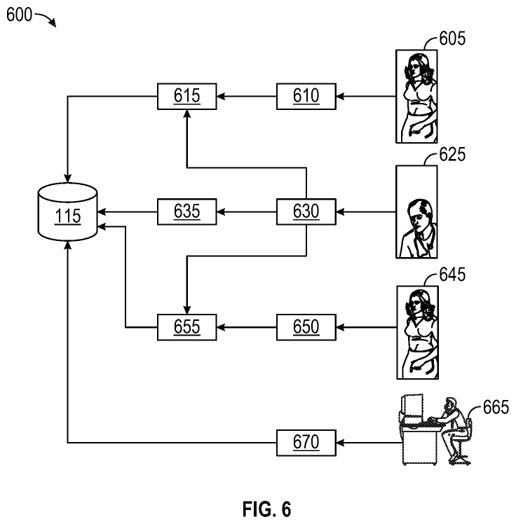

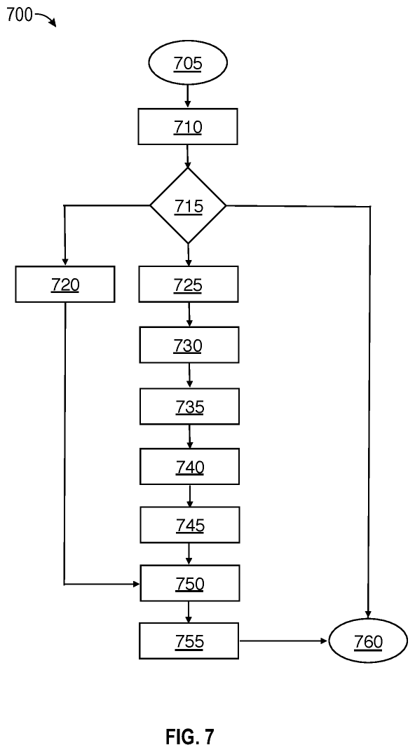

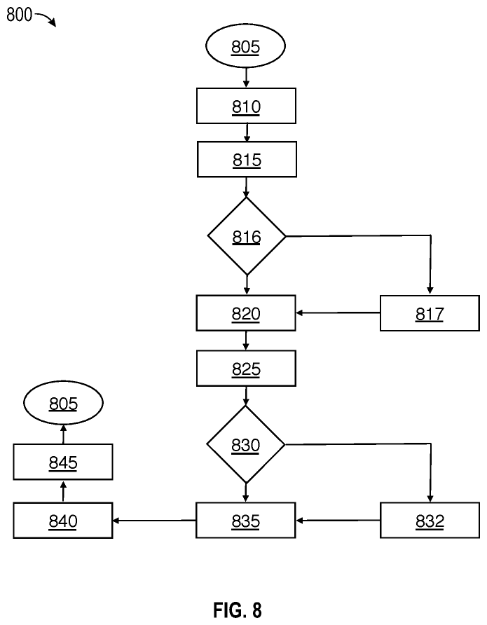

FIGS. 4-8 illustrate embodiments of a system 400 for managing information related to a patient's medical history. As shown in FIG. 4, the system 400 generally comprises a computing entity 200 having a user interface 411, a processor 220, a non-transitory computer-readable medium 416 coupled to the processor 220 and having instructions stored thereon, and a database 115 operably connected to the processor 220 and having patient health records 430 stored therein. A display 316 may present information of the user interface 411 to the healthcare professional 405. In another embodiment, a server 110 may be operably connected to the database 115 and processor 220, facilitating the transfer of information between the processor 220 and database 115. It is understood that the various method steps associated with the methods of the present disclosure may be carried out as operations by the system 400 shown in FIG. 4. FIG. 4 illustrates an embodiment of the disclosed system 400. FIG. 5 illustrates example screenshots of a user interface 411 of the system 400. FIG. 6 illustrates permission levels 600 that may be utilized by the present system 400 for controlling access to patient health records 430 and event data 437. FIGS. 7 and 8 illustrate the various methods that may be carried out by the system 400.

The system 400 may deconstruct patient health records 430 into event data 437 that may be transformed into medical events 439. In one preferred embodiment, event data 437 and medical events 439 may be stored in patient profiles 435 of the system 400. Because patient health records 430 often contain chronological data 440 within, event data 437 is preferably associated with chronological data 440 after deconstruction so that the system 400 may chronologically order a patient's medical history within a patterned timeline 505. The processor 220 may transform event data 437 and/or chronological data 440 into medical events 439 that may be used to create a visual representation of a patient's medical history within the user interface 411 and present it to a healthcare professional 405 via a display 316. In a preferred embodiment, patient health records 430 comprise of the EHR, wherein the EHR is defined as a system 400 [symbol] atized collection of patient and population electronically-stored health information in a digital format. Deconstruction of patient health records 430 involves parsing the information within patient health records 430 and categorizing the resulting data in a way such that the system 400 may transform that data into medical events 439.

After the system 400 deconstructs a patient's patient health records 430, the system 400 may reassemble the event data 437 and/or chronological data 440 to create medical events 439. In a preferred embodiment, the event data 437 may consist of 240 characters or less of text that convey the most important aspects of a particular patient health record; however, more characters may be used without departing from the inventive subject matter described herein. These characters may be viewed by a healthcare professional 405 within the user interface 411 when a healthcare professional 405 selects a medical event 439 of the patterned timeline 505. In some preferred embodiments, the system 400 may link the patient health record to the medical event 439 within the user interface 411 in a way such that a healthcare professional 405 may quickly access the full patient health record if needed. Therefore, a medical event 439 contains a summary of the information within a particular patient health record as well as other information that may be useful to a healthcare professional 405 when reviewing a patient's medical history. The medical events 439 may be ordered on a patterned timeline 505 of the user interface 411 in a way such that it may allow a healthcare professional 405 to easily review a patient's medical history. In a preferred embodiment, the system 400 may visualize medical events 439 within the user interface 411 chronologically in a way that allows the healthcare professional 405 to quickly gain insight into a patient's medical history without searching each file of a patient within the EHR. In another preferred embodiment, event data 437, chronological data 440, and medical events 439 may be stored within a patient profile of the database 115.

The system 400 may use machine learning techniques to parse patient health records 430. For instance, pattern recognition or computer-aided detection may be used to remove the pertinent information of each patient record in the EHR before creating medical events 439 and subsequently presenting it to the healthcare professional 405. The system 400 may use more than one machine learning technique to determine which categories patient data 615, 635, 655 should be placed. Machine learning techniques may also be used to as assist healthcare professionals 405 in making diagnoses. For instance, the system 400 may use supervised deep learning combined with results from computer-aided detection and compound term processing to better assist a healthcare professional 405 in making diagnoses based on the parsed data and pattern in which the data is presented. Over time, the system 400 may obtain more knowledge and be able to become more useful in helping healthcare professionals 405 make an accurate diagnosis based on the created pattern.

Alternatively, the system 400 may assist a healthcare professional 405 in a way that does not involve the use of machine learning techniques. In one preferred embodiment, the system 400 may use indicia to highlight patient data 615, 635, 655 that may be relevant to a patient's medical history. For instance, a patient undergoing chemo therapy might be highlighted about the patterned timeline 505 in a way that indicates the patient is undergoing chemotherapy treatment while the pattern of the data about the patterned timeline 505 may indicate to the healthcare professional 405 how far along said patient is in the chemotherapy treatment regimen. For instance, a patient may have a series of seemingly unrelated symptoms throughout their lifetime that taken together may indicate a certain medical condition based on the pattern, shape, and colors of the presented medical history. The system 400 may be programmed to search for certain key words and alert a healthcare professional 405 via a message or indicia to be mindful of certain symptoms experienced by the patient due to the possible presence of a certain medical condition or to instruct the healthcare professional 405 to perform certain tests (such as blood tests) on the patient. In another preferred embodiment, the system 400 may use digital twins, which may allow the system 400 to simulate various scenarios of a patient's future medical events 439. For instance, the system 400 may create digital twins for a patient that shows what medical events 439 the patient may expect in their future should they elect to enter a drug trial or not enter a drug trial. This would allow a healthcare professional 405 to provide a patient with multiple detailed scenarios of treatment for a condition.

In an embodiment, the programming instructions responsible for the operations carried out by the processor 220 are stored on a non-transitory computer-readable medium 416f (“CRM”), which may be coupled to the server 110, as shown in FIG. 4. Alternatively, the programming instructions may be stored or included within the processor 220. Examples of non-transitory computer-readable mediums 416 include, but are not limited to, magnetic media such as hard disks, floppy disks, and magnetic tape; optical media such as CD ROM discs and DVDs; magneto-optical media such as optical discs; and hardware devices that are specifically configured to store and perform programming instructions, such as read-only memory (ROM), random access memory (RAM), flash memory, and the like. In some embodiments, the programming instructions may be stored as modules within the non-transitory computer-readable medium 416.

In an embodiment, the system 400 may further comprise a computing entity 200 operably connected to the processor 220. A computing entity 200 may be implemented in a number of different forms, including, but not limited to, servers 110, multipurpose computers, mobile computers, etc. For instance, a computing entity 200 may be implemented in a multipurpose computer that acts as a personal computer for a healthcare professional 405, such as a laptop computer. For instance, components from a computing entity 200 may be combined in a way such that a mobile computing device 350 is created, such as mobile phone. Additionally, a computing entity 200 may be made up of a single computer or multiple computers working together over a network. For instance, a computing entity 200 may be implemented as a single server 110 or as a group of servers 110 working together over and Local Area Network (LAN), such as a rack server 110 system. Computing entities may communicate via a wired or wireless connection. For instance, wireless communication may occur using a Bluetooth, Wi-Fi, or other such wireless communication device.

In an embodiment, the system 400 may further comprise a user interface 411. A user interface 411 may be defined as a space where interactions between a healthcare professional 405 and the system 400 may take place. In a preferred embodiment, the interactions may take place in a way such that a healthcare professional 405 may control the operations of the system 400, and more specifically, allow a healthcare professional 405 to control how the system 400 transforms event data 437 into a visual representation of a patient's medical history. A healthcare professional 405 may input instructions to control operations of the system 400 manually using an input device. For instance, a healthcare professional 405 may choose data filters 510 or pattern parameters 515 where directed by the user interface 411, as illustrated in FIG. 5, using an input device, including, but not limited to, a keyboard, mouse, or touchscreen. A user interface 411 may include, but is not limited to operating systems, command line user interfaces, conversational interfaces, web-based user interfaces, zooming user interfaces, task-based user interfaces, touch user interfaces, text-based user interfaces, intelligent user interfaces, and graphical user interfaces, or any combination thereof. The system 400 may present data of the user interface 411 to the healthcare professional 405 via a display 316 operably connected to the processor 220.

A display 316 may be defined as an output device that communicates data that may include, but is not limited to, visual, auditory, cutaneous, kinesthetic, olfactory, and gustatory, or any combination thereof. Information presented via a display 316 may be referred to as a soft copy of the information because the information exists electronically and is presented for a temporary period of time. Information stored on the non-transitory computer-readable medium 416 may be referred to as the hard copy of the information. For instance, a display 316 may present a soft copy of a visual representation of a patient's medical history via a liquid crystal display 316 (LCD), wherein the hardcopy of the visual representation of a patient's medical history may be stored on a local hard drive. For instance, a display 316 may present a soft copy of audio information via a speaker, wherein the hard copy of the audio information is stored on a flash drive. For instance, a display 316 may present a soft copy of a patient's medical history pattern, wherein the hard copy of the timeline containing a patient's medical history pattern is stored within a database 115. Displays 316 may include, but are not limited to, cathode ray tube monitors, LCD monitors, light emitting diode (LED) monitors, gas plasma monitors, screen readers, speech synthesizers, haptic suits, speakers, and scent generating devices, or any combination thereof, but is not limited to these devices.

As shown in FIG. 4, the system 400 may comprise a database 115 operably connected to the processor 220. The database 115 may be operably connected to the processor 220 via wired or wireless connection. In a preferred embodiment, the database 115 is configured to store patient health records 430, patient profiles 435, event data 437, medical events 439, and chronological data 440 therein. The database 115 may be a relational database such that the patient health records 430, patient profiles 435, event data 437, and medical events 439, and chronological data 440 associated with each patient profile within the plurality of patient profiles 435 may be stored, at least in part, in one or more tables. Alternatively, the database 115 may be an object database such that the patient health records 430, patient profiles 435, event data 437, medical events 439, and chronological data 440 associated with each patient profile within the plurality of patient profiles 435 are stored, at least in part, as objects. In some instances, the database 115 may comprise a relational and/or object database and a server 110 dedicated solely to managing the patient health records 430 and patient profiles 435 containing event data 437, medical events 439, and chronological data 440 in the manners disclosed herein.

As mentioned previously, the system 400 may comprise a power supply. The power supply may be any source of power that provides the system 400 with electricity. In one preferred embodiment, the system 400 may comprise of multiple power supplies that may provide power to the system 400 in different circumstances. For instance, the system 400 may be directly plugged into a stationary power outlet, which may provide power to the system 400 so long as it remains in one place. However, the system 400 may also be connected to a battery so that the system 400 may receive power even when the it is not connected to a stationary power outlet. In this way, the system 400 may always receive power so that it may continuously update patient profiles 435 and provide healthcare professionals 405 with a patterned timeline 505 of a patient's medical history.

In a preferred embodiment, healthcare professional 405 may access patient health records 430, patient profiles 435, event data 437, medical events 439, and chronological data 440 via the user interface 411, which may be accomplished by causing the processor 220 to query the non-transitory computer-readable medium 416 and/or database 115 for relevant patient data 615, 635, 655. The non-transitory computer-readable medium 416 and/or database 115 may then transmit the patient health records 430, patient profiles 435, event data 437, medical events 439, and chronological data 440 back to the processor 220, wherein the processor 220 may then transform and/or organize the data before presenting it to a healthcare professional 405 via a display 316. In a preferred embodiment, the user interface 411 may present a particular patient's medical history to a healthcare professional 405 using a patterned timeline 505. This patterned timeline 505 may convey to the healthcare professional 405 a patient's medical history in a way such that the healthcare professional 405 may quickly review medical events 439 the patient has experienced in their lifetime, which may allow the healthcare professional 405 to more efficiently address a patient's needs. For instance, the system 400 may present past medical events 439 of a patient about a patterned timeline 505 in way that may quickly indicate to the healthcare professional 405 that said patient is currently in remission for breast cancer. In a preferred embodiment, this may be done by dimensionally positioning medical events 439 about the timeline. For instance, the system 400 may present medical events 439 that happened within a 24-hour period from one another horizontally about the patterned timeline 505; present medical events 439 that happened in a time period greater than 24 hours and less than one week from one another diagonally about the patterned timeline 505; and present medical events 439 that happened in a time period greater than a week from one another vertically about the patterned timeline 505.

In another preferred embodiment, the system 400 may use indicia to modify a patterned timeline 505 in a way such that it conveys additional information to a healthcare professional 405. In a preferred embodiment, the color in which the medical events 439 are highlighted on said patterned timeline 505 may change depending on information obtained about a medical event 439 by the system 400 during the parsing process. This may convey information about a medical event 439 that dimensional spacing about the patterned timeline 505 may not. For instance, medical events 439 may be highlighted with a certain color based on the type of symptom the patient received treatment for. For instance, medical events 439 highlighted in red may represent medical events 439 in which a patient was treated for symptoms pertaining to cardiovascular disease. For instance, the system 400 may highlight event data 437 pertaining to subjective information in orange, objective information in blue, assessment information in red, and plan information in green. Alternatively, the system 400 may highlight medical events 439 based on the type of treatment received by the patient, which may be based on information obtained by the system 400 when parsing the files of a patient within the HER prior to the creation of the medical event 439. For instance, a medical event 439 may be highlighted in purple about a patterned timeline 505 to quickly convey to a doctor that a patient received chemotherapy on a particular date.

Healthcare professionals 405 may operate the user interface 411 to acquire the patient's health records 430 from the database 115 and have them transformed into a visual representation of the patient's medical history that may be viewed with the user interface 411. In a preferred embodiment, a healthcare professional 405 may operate the user interface 411 in a way that allows the healthcare professional 405 to change how the system 400 generates a patterned timeline 505 of medical events 439 of a patient. In one preferred embodiment, a healthcare professional 405 may choose pattern parameters 515 that change how a timeline may be patterned so that the patterned timeline 505 best suits the healthcare professional's 405 needs. For instance, the patterned timeline 505 an oncologist may review prior to meeting a patient to discuss post chemotherapy results may differ from the desired patterned timeline 505 a family practice physician may review prior to meeting a patient for an annual checkup. In a preferred embodiment, a pattern parameter 515 instructs the processor 220 how to structure patterned timelines 505 based on variables and sub-variables. Variables and sub-variables that may be used as pattern parameters 515 include, but are not limited to, direction of events 515A, time between visits 515B, color 515C, symbols 515D, dimensionality 515E, scale 515F, or any combination thereof.

In another preferred embodiment, a healthcare professional 405 may choose a category or sub-category, which may instruct the system 400 to focus on particular types of medical maladies throughout a patient's lifetime using data filters 510. A data filter 510 is a function that allows a healthcare professional 405 to modify what data the system 400 may choose when determining the data used to create a patterned timeline 505. In a preferred embodiment, a data filter 510 allows the processor 220 to restrict the search for medical events 439 to certain categories. Categories or sub-categories that may be used as data filters 510 include, but are not limited to, heart disease 510A, diabetes 510B, cancer 510C, psychological disorders 510D, autoimmune disorders 510E, infectious diseases 510F, or any combination thereof. For instance, a healthcare professional 405 may select a variable or sub-variable entitled “abdominal disorders” within the data filters 510 section of the user interface 411 when performing a medical evaluation on a patient complaining of abdominal pain. The system 400 may then search for data within the patient's patient profile related to abdominal issues.

To prevent un-authorized users from accessing a patient's sensitive medical information, the system 400 may employ a security method. As illustrated in FIG. 6, the security method of the system 400 may comprise a plurality of permission levels 600 that may allow a healthcare professional 405 to view patient data 615, 635, 655 within the database 115 while simultaneously denying users without appropriate permission levels 600 the ability to view patient data 615, 635, 655. To access the patient data 615, 635, 655 stored within the database 115, healthcare professionals 405 may be required to make a request via a user interface 411. Access to the data within the database 115 may be granted or denied by the processor 220 based on verification of a requesting user's 605, 625, 645 permission level 600. If the requesting user's 605, 625, 645 permission level 600 is sufficient, the processor 220 may provide the requesting user 605, 625, 645 access to patient data 615, 635, 655 stored within the database 115. Conversely, if the requesting user's 605, 625, 645 permission level 600 is insufficient, the processor 220 may deny the requesting user 605, 625, 645 access to patient data 615, 635, 655 stored within the database 115. In an embodiment, permission levels 600 may be based on user roles 610, 630, 650 and administrator roles 670, as illustrated in FIG. 6. User roles 610, 630, 650 allow users to access patient data 615, 635, 655 that a healthcare professional 405 has uploaded and/or otherwise obtained through use of the system 400. Administrator roles 670 allow administrators 665 to access system 400 wide data.

In an embodiment, user roles 610, 630, 650 may be assigned to a healthcare professional 405 in a way such that a requesting user 605, 625, 645 may access patient health records 430 and patient profiles 435 containing event data 437, medical events 439, and chronological data 440 via a user interface 411. In an embodiment, the system 400 may be configured to send a healthcare professional 405 a notification indicating that another healthcare professional 405 has shared information with them. To access the data within the database 115, a healthcare professional 405 may make a user request via the user interface 411 to the processor 220. In an embodiment, the processor 220 may grant or deny the request based on the permission level 600 associated with the requesting user 605, 625, 645. Only healthcare professionals 405 having appropriate user roles 610, 630, 650 or administrator roles 670 may access the patient health records 430. For instance, as illustrated in FIG. 6, requesting user 1 605 has permission to view patient 1 data 615 whereas requesting user 2 625 has permission to view patient 1 data 615, patient 2 data 635, and patient 3 data 655. Alternatively, patient data 615, 635, 655 may be restricted in a way such that a healthcare professional 405 may only view a limited amount of patient data 615, 635, 655. For instance, requesting user 3 645 may be granted a permission level 600 that only allows them to view patient 3 data 655 related to medications. Therefore, the permission levels 600 of the system 400 may be assigned to healthcare professionals 405 in various ways without departing from the inventive subject matter described herein.

FIG. 7 provides a flow chart 700 illustrating certain, preferred method steps that may be used to carry out the method for deconstructing patient health records 430 into medical events 439 so that it may be transformed into a patterned timeline 505. Step 705 indicates the beginning of the method. During step 710, the processor 220 may query the non-transitory computer-readable medium 416 and/or database 115 for a patient profile containing event data 437 and/or medical events 439 or patient health records 430. In a preferred embodiment, the processor 220 first may check the database 115 for patient profiles 435 containing event data 437. If no event data 437 is present, the processor 220 may check for patient health records 430 within the EHR. Based on the results of the query, the processor 220 may determine the appropriate course of action to take in step 715. If processor 220 determines that a patient has no patient profile containing event data 437 and/or medical events 439 or patient health records 430 within the non-transitory computer-readable medium 416 and/or database 115, the system 400 may proceed to the terminate method step 760. If the processor 220 determines that a patient has a patient profile containing event data 437 and/or medical events 439 within the non-transitory computer-readable medium 416 and/or database 115, the processor 220 may retrieve that data from the non-transitory computer-readable medium 416 and/or database 115 in step 720 and then proceed to step 750. If the processor 220 determines that a patient does not have a related patient profile containing event data 437 and/or medical events 439 but does have patient health records 430 within the non-transitory computer-readable medium 416 and/or database 115, the processor 220 may retrieve the patient health records 430 during step 725.

Once the patient health records 430 have been retrieved, the processor 220 may deconstruct the patient health records 430 into event data 437 during step 730. In a preferred embodiment, the event data 437 may consist of 240 characters or less of text that convey the most important aspects of a particular patient health record. The system 400 may create a patient profile using said event data 437 during step 735 and may subsequently create a user profile during step during step 740. The event data 437 may be saved within the patient profile during step 745. Once the patient profile and event data 437 have been saved, the processor 220 may transform the event data 437 into a medical event 439 during step 750. Once the event data 437 has been transformed into a medical event 439 by the processor 220, the processor 220 may display 316 the medical event 439 within the user interface 411 via a display 316 during step 755. In a preferred embodiment, the medical events 439 are presented about a patterned timeline 505 within the user interface 411. The method may proceed to the terminate method step 760 after presenting the medical events 439.

FIG. 8 provides a flow chart 800 illustrating certain, preferred method steps that may be used to carry out the method of plotting medical events 439 about a patterned timeline 505. Step 805 indicates the beginning of the method. During step 810, the processor 220 may receive event data 437 from the database 115 and/or non-transitory computer-readable medium 416. During step 815, the processor 220 may perform a query to determine if a data filter 510 has been selected within the user interface 411, which may instruct the processor 220 to restrict the search for event data 437 so that only event data 437 meeting certain search parameters is returned. Based on the results of the query, the processor 220 may take an action during step 816. If the processor 220 determines no data filter 510 has been selected, the processor 220 may analyze the event data 437 in step 820. If the processor 220 determines that a data filter 510 has been selected within the user interface 411, the processor 220 may proceed to filter the event data 437 in step 817. In a preferred embodiment, the processor 220 may filter the event data 437 by limiting the search for event data 437 to only those categories selected by a healthcare professional 405. For instance, a healthcare professional 405 who chooses the “cancer related treatment” filters may only have event data 437 returned related to the selected categories and subcategories. For instance, a healthcare professional 405 who chooses a data filter 510 pertaining to the subcategory of “heart disease” may only have event data 437 returned that is related to heart disease. For instance, a healthcare professional 405 who chooses a data filter 510 instructing the processor 220 to not search for event data 437 related to “infectious diseases” may have all event data 437 returned except for that which pertains to infectious diseases.

Once the event data 437 has been filtered, the method may proceed to step 820, wherein the processor 220 may transform the event data 437 into a medical event 439. The system 400 may then perform a query to determine if any pattern parameters 515 have been selected within the user interface 411 during step 825. In a preferred embodiment, medical events 439 are assigned a spatial position about the patterned timeline 505 based on the amount of time between medical events 439. In one preferred embodiment, a healthcare professional 405 may alter how the system 400 assigns a spatial position as well as the amount of time required between medical events 439 before that spatial position is assigned. In a preferred embodiment, medical events 439 are ordered chronologically and assigned a spatial position according to any associated chronological data 440. In another preferred embodiment, a healthcare professional 405 may alter indicia used by the system 400 to indicate information about a medical event 439 using pattern parameters 515. For instance, a healthcare professional 405 may alter the color of the medical event 439 about the timeline in a way that indicates the medical specialty in which the medical event 439 was most related.

The processor 220 may take an action based on the results of the query during step 830. If the processor 220 determines that a pattern parameter 515 has been selected, the processor 220 may apply the pattern parameter 515 prior to constructing the patterned timeline 505 during step 832 and subsequently proceed to step 835. If the processor 220 determines that no pattern parameter 515 has been selected, the processor 220 may proceed to step 835, wherein medical events 439 may be mapped about a patterned timeline 505 during step 840. Once the patterned timeline 505 has been created, the patterned timeline 505 may be presented via display 316 during step 845. Once the patterned timeline 505 has been presented via the display 316, the method may proceed to the terminate method step 850.

The subject matter described herein may be embodied in systems, apparati, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one input/output device.

These computer programs, which may also be referred to as programs, software, applications, software applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display device, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For example, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and method stages which have been described and illustrated in order to explain the nature of this inventive subject matter can be made without departing from the principles and scope of the inventive subject matter.

What is claimed is:

1) A system for transforming information of the electronic health record into a patterned timeline comprising:
a processor operably connected to a computing entity,
wherein said computing entity hosts a user interface used to access patient health records and visualize medical events,
a power supply,
a display operably connected to said processor,
a non-transitory computer-readable medium coupled to said processor and having instructions stored thereon, which, when executed by said processor, cause said processor to perform operations comprising:
acquiring said patient health records,
parsing said patient health records into event data,
creating said medical events from said event data,
creating a patterned timeline using said medical events,
presenting said patterned timeline via said display.

2) The system of claim 1, wherein said non-transitory computer-readable medium stores data transmitted to and from said processor.

3) The system of claim 2, further comprising additional instructions stored on said non-transitory computer-readable medium, which, when executed by said processor, cause said processor to perform additional operations comprising:
querying said non-transitory computer-readable medium for patient profiles containing said medical events, and
receiving said medical events from said non-transitory computer-readable medium.

4) The system of claim 1, further comprising a database operably connected to said processor,
wherein said database receives said patient health records and said medical events transmitted to said processor and stores it within said patient profiles.

5) The system of claim 1, further comprising an input device,
wherein said input device is used to input instructions into said user interface.

6) The system of claim 5, further comprising additional instructions stored on said non-transitory computer-readable medium, which, when executed by said processor, cause said processor to perform additional operations comprising:
creating said patterned timeline within said user interface using pattern parameters,
wherein said pattern parameters determine a spatial position of said medical events about said timeline based on chronological data,
wherein said pattern parameters symbolically alter said medical events about said timeline using indicia,
analyzing said medical events to determine said spatial position and said indicia.

7) The system of claim 1, further comprising additional instructions stored on said non-transitory computer-readable medium, which, when executed by said processor, cause said processor to perform additional operations comprising:
filtering said medical events using at least one data filter,
wherein said at least one data filter comprises a plurality of categories,
wherein performing a search while using said at least one data filter narrows said search according to which said at least one data filters have been selected. 8) The system of claim 1, further comprising additional instructions stored on said non-transitory computer-readable medium, which, when executed by said processor, cause said processor to perform additional operations comprising:
creating said patterned timeline using chronological data of said medical events,
wherein said patterned timeline has a beginning date matching an oldest chronological date and an ending date matching a newest chronological date, and
mapping said medicals events about said patterned timeline using said chronological data.

9) A method for transforming electronic health record data into a patterned timeline comprising the steps of:
acquiring a computing device with the ability to create patterned timelines,
searching for patient health records,
acquiring said patient health records,
creating medical events from said patient health records,
creating said patterned timeline using said medical events,
presenting said patterned timeline via said display.

10) The method of claim 9, wherein said method further comprises the step of mapping said medical events about said patterned timeline using chronological data.

11) The method of claim 9, wherein said method further comprises the step of determining a spatial position of said medical event, wherein said spatial position is determined by pattern parameters.

12) The method of claim 11, wherein said method further comprises the step of using chronological data to determine said spatial position about said patterned timeline.

13) The method of claim 11, wherein said method further comprises the step of determining indicia of said medical event, wherein said indicia is determined by pattern parameters.

14) The method of claim 9, wherein said method further comprises the step of filtering said medical events using at least one data filter, wherein said at least one data filter comprises a plurality of categories, wherein performing a search while using said at least one data filter narrows said search according to which said at least one data filters have been selected.

15) A method for transforming electronic health record data into a patterned timeline comprising the steps of:
searching for a patient profile containing medical events,
acquiring said medical events from said patient profile,
creating a patterned timeline using said medical events,
presenting said patterned timeline via a display.

16) The method of claim 15, wherein said method further comprises the step of mapping said medical events about said patterned timeline using chronological data.

17) The method of claim 15, wherein said method further comprises the step of determining a spatial position of said medical event, wherein said spatial position is determined by pattern parameters.

18) The method of claim 17, wherein said method further comprises the step of using chronological data to determine said spatial position about said patterned timeline.

19) The method of claim 17, wherein said method further comprises the step of determining indicia of said medical event, wherein said indicia is determined by pattern parameters.

20) The method of claim 15, wherein said method further comprises the step of filtering said medical events using at least one data filter, wherein said at least one data filter comprises a plurality of categories, wherein performing a search while using said at least one data filter narrows said search according to which said at least one data filters have been selected.
